(19) United States
(12) Patent Application Publication
(10) Pub. No.: US 2012/0288850 A1
(43) Pub. Date: Nov. 15, 2012

(54) METHODS FOR DIAGNOSIS OF IMMUNE RESPONSES AGAINST VIRUSES

(75) Inventors: Ernst Christiaan Soethout, Odijk (NL); Wai Ming Liu, Utrecht (NL)

(73) Assignee: De Staat Der Nederlanden, Vert. Door De Minister Van VWS, The Hague (NL)

(21) Appl. No.: 13/503,000

(22) PCT Filed: Oct. 21, 2010
(86) PCT No.: PCT/NL2010/050699

§ 371 Date: Jul. 16, 2012

Related U.S. Application Data
(60) Provisional application No. 61/253,598, filed on Oct. 21, 2009.

(30) Foreign Application Priority Data
Oct. 21, 2009 (EP) 09173668.6

(51) Int. Cl. C12Q 1/70

(52) U.S. Cl. 435/5

(57) ABSTRACT

The present invention relates to methods for diagnosis of a cellular immune responses against a virus using an inactivated virus. In the method of the invention a cellular immune response against the virus is detected in a subject by incubating PBMCs from the subject with a preparation of inactivated virus and subsequently detecting the expression of at least one T cell specific cytokine in the subject's PBMCs, preferably by flow cytometry. Advantageously in the method, inactivated virus is used for incubation with PBMCs from the subject so as to make feasible that the method is performed in laboratories without BSL-3 classification. Preferably the method is a method for detecting a CD4+ and/or CD8+ T cell response against an influenza virus by detecting expression of one or more of CD107, IFN-γ, IL-2, IL-10 and TNF-α, using formalin inactivated influenza virus. The invention further pertains to kits comprising components that are useful for detecting a cellular immune responses in a subject against a virus.

### FIELD OF THE INVENTION

The present invention relates to the field of medicine. In particular the invention relates to the fields of virology and immunology, more particularly the invention relates to methods for diagnosis of cellular immune responses against a virus using an inactivated virus, such as influenza virus.

### BACKGROUND OF THE INVENTION

Performing studies on immune responses against highly pathogenic and pandemic influenza viruses requires a BSL-3 high containment facility. The constraints in such a facility substantially affect laboratory potential. Therefore, there is need for assays able to detect immune responses against highly pathogenic influenza viruses that can be performed without the need of a BSL-3 high containment facility.

By inactivating highly pathogenic and pandemic influenza, these viruses can be used in laboratories without BSL-3 classification. Currently, during the production of split- and subunit influenza vaccines chemical treatment with formalin is applied to inactivate influenza virus and can also be used for inactivating whole virus influenza (1, 2). Formalin acts by irreversibly cross-linking proteins, and thereby completely removing viral infectivity (3). Therefore, this procedure efficiently inactivates influenza viruses and allows the use of formalin-treated inactivated virus outside BSL-3 constraints.

Humoral immune responses against influenza are determined by standard hemagglutination- and neuraminidase inhibition assays (4, 5, 12). Detection of cellular immune responses against inactivated influenza virus however, has shown to be difficult. CTL activity as determined by conventional ⁵¹Cr-release assay lacks sensitivity (6). The expansion of memory CTLs during a seven day in vitro stimulation before the ⁵¹Cr-release assay possibly masks any differences, indicating that in vitro expansion reduces the sensitivity of the assay. A more sensitive assay like the ELISPOT assay is able to detect secreted cytokines produced by cells that are in the immediate vicinity, and does not require an in vitro expansion procedure (7). Although the ELISPOT is sensitive, this assay is not able to detect multiple effector functions at the same time and is not able to distinguish distinct functional populations.

Importantly, T cells having multiple effector functions (multifunctional T cells), i.e. producing multiple cytokines and/or cytotoxic agents, have been shown to be important for determining the quality of the immune response. Hence, detection of these cells by flow cytometry facilitates determining the quality of a vaccine and for vaccine design (8).

Rutebemberwa et al. (9) discloses a method of inactivating HIV-1 with Aldrithiol. PBMCs are then isolated and exposed to the inactivated virus and flow cytometry is performed in order to detect, inter alia, IFN-gamma.

Skarsvik et al. (10) discloses how PBMCs were stimulated with heat inactivated Coxsackie virus followed by flow cytometric analysis for detection of T cell activation with several markers.

Betts et al. (11) disclose stimulation of PBMCs with a cocktail of peptides which represent antigenic parts of HIV-1 followed by flow cytometric analysis for detection of T cell activation with several markers. However, the use of overlapping peptides is not a valid alternative for determining the complete physiological relevant CD8 T cell responses against whole live viruses. It is practically impossible to generate banks of overlapping peptides covering the complete viral proteome and at the same time have enough PBMCs for stimulation with these banks. In addition, the exogenous addition of the peptides excludes intracellular processing of the peptides prior to presentation by MHC molecules and thereby responses dependent on intracellular post translational modification may be missed. Furthermore, natural processing, allows presentation of peptides at natural levels which will lead to physiologically relevant in vitro immune responses, whereas exogenous addition of peptides may lead to stimulation of physiologically irrelevant T cell responses.

The prior art does, however, not disclose a method for detecting a T cell response against a virus using inactivated virus, whereby the T cell response reliably reflects the response obtained with live virus with sufficient intensity. In particular the prior art does not disclose such methods for detecting T cell response against influenza virus.

There is therefore still a need in the art to provide for means and methods for reliable and sensitive simultaneous detection of multiple markers and for phenotyping T cell responses against viruses, particularly influenza virus, with respect to the number and type of effector functions as well as expression of distinct markers. It is an object of the present invention to provide for such means and methods.

### DESCRIPTION OF THE INVENTION

A complicating factor in the detection of immune response against viruses is that usage of live virus is generally seen as an absolute necessity for determining CD8⁺ T cell responses, as only live virus infection of T cells allows expression of viral epitopes on MHC-class I molecules, through the direct presentation pathway (K. L. Rock, The Journal of Immunology, 2010, 184, 9-15). However, because live virus infection requires BSL-3 conditions for highly pathogenic viruses, we have developed assays that allow detection of CD8 T cell responses after stimulation with inactivated virus. This technology makes use of an alternative route of presentation that is unique for dendritic cells and macrophages to present on MHC class I peptides from antigens in their external environment through a process called cross-presentation (K. L. Rock, The Journal of Immunology, 2010, 184, 9-15).

In a first aspect the present invention relates to a method for detecting the presence of an immune response against a virus in a subject. The method preferably is a method for detecting a cellular immune response against the virus. More particularly the method detects a T cell response against the virus. The method for detecting an immune response against a virus in a subject preferably comprises the steps of: a) incubating PBMCs from the subject with inactivated virus; and, b) detecting the expression of at least one marker for T cell activity in the PBMC by flow cytometry.

Advantageously in the method, inactivated virus is used for incubation with PBMCs from the subject so as to make feasible that the method is performed in laboratories without BSL-3 classification. Various means for inactivation of viruses are known in the art. E.g. chemical means for inactivating a virus include treatment with an effective amount of one or more of the following agents: detergents (e.g. Triton X-100), formaldehyde, formalin, beta-propiolactone, or merthiolate. Additional chemical means for inactivation may include treatment with methylene blue, psoralen, carboxyfullerene (C60) or a combination of any thereof. Other methods of viral inactivation are known in the art, such as for example binary ethylamine, acetyl ethyleneimine, and physical means such as heat, UV light or gamma irradiation. For the preparation of inactivated virus for use in the methods of the present invention any of these means or combinations thereof may be applied.

An inactivated virus is herein understood to mean a viral preparation with preferably no detectable infectivity. Infectivity of preparations of inactivated virus for use in the present invention may be determined using methods well known in the art (see e.g. Goldstein and Tauraso, 1970, supra). Generally, the infectivity of an inactived preparation is compared to the infectivity of a corresponding non-treated and/or mock-treated viral preparation whereby the titer of the inactivated viral preparation is at least 6, 7, 8, 9, or 10 orders of magnitude lower than the titer of the non-treated and/or mock-treated viral preparation. Preferably in the inactivated preparation no viral infectivity can be detected.

In a preferred method for preparing inactivated virus for use in the present invention the inactivated virus is obtained by treatment of the virus for at least 18 hours with at least about 0.02% v/v formalin. Preferably, the virus is treated at 37° C. The skilled person will understand that the minimum inactivation time and the formalin concentration may be varied, e.g. a longer inactivation time will be required when using a lower formalin concentration and a shorter time may be required at a higher formalin concentration. Thus, in a preferred method for preparing inactivated virus for use in the present invention the inactivated virus is obtained by a formalin treatment of the virus under conditions (i.e. for a period of time, at a concentration of formalin and at a temperature) that produces the same degree of inactivation (i.e. the titer of inactivated viral preparation being the same order of magnitude lower than the titer of the same viral preparation that is not treated and/or mock-treated) as obtained by treatment of the virus for at least 18 hours with at least about 0.02% v/v formalin at 37° C.

In a further preferred method for preparing inactivated virus for use in the present invention the inactivated virus, the inactivating agent (e.g. formaldehyde) is removed from the preparation of inactivated virus by processes such as dialysis, diafiltration, or chromatography, e.g. size-exclusion or affinity chromatography. Means and methods for such processes are well known in the art. It will be clear that inactivation of the viral preparation will be performed in laboratories with the required BSL classification. However, after inactivation and removal of inactivating agent the inactivated viral preparation may be transported to and stored and/or used in accordance with the present invention in laboratories without BSL-3 classification.

In the method of the invention the inactivated virus is incubated with PBMCs. PBMCs, (Peripheral Blood Mononuclear Cells) may be obtained from human or other mammalian blood using a variety of methods well known in the art (see e.g. Coligan et al., 1994, In: Coico R, ed. Current protocols in immunology. Vol. 2: John Wiley & Sons, Inc., 1994:711-2.). The composition comprising PBMCs may e.g. consist of the PBMC bulk that is obtainable by aphaeresis, Ficoll density gradient centrifugation and/or red blood cell lysis from mammalian or human blood.

In the method of the invention PBMCs and inactivated virus are incubated in medium and under condition appropriate for maintenance, proliferation and/or stimulation of T cells as are well known in the art (see e.g. Current protocols in immunology, Coico R, ed, John Wiley & Sons, Inc., 1994). In the methods of the invention, PBMCs and inactivated virus are preferably incubated between about 24 and 96 hours before detection of cytokine expression by flowcytometry. Thus, in the method if the invention, cytokine expression is preferably detected after between about 24 and 96 hours of incubation of the PBMCs with the virus, i.e. between about 24-96 hours post “infection” or rather post-stimulation. More preferably, cytokine expression is detected after at least 36, 48, 60, 66, 68, or 70 hours of incubation of the PBMCs with the virus and preferably after no more than 96, 90, 84, 78 or 74 hours of incubation of the PBMCs with the virus. Most preferably, cytokine expression is detected after around 72 hours of incubation of the PBMCs with the virus.

In one embodiment of the method of the invention, the expression of at least two different markers for T cell activity is detected. Preferably, in the method the expression of at least two different markers for T cell activity is detected simultaneously, e.g. by using at least two different fluorescent labels for (simultaneous) the detection of each marker for T cell activity.

Generally in the methods of the invention markers for T cell activity and/or differentiation antigens (e.g. CD4 or CD8) may suitably be detected in flow cytometry by using (monoclonal) antibodies specific for the marker or antigen to be detected, which antibodies are labeled by conjugation to a fluorescent label, i.e. fluorophores. A large variety of fluorescent labels is available to the skilled person for conjugation to antibodies against the cytokines or differentiation antigens to be detected. Suitable fluorophores that may be conjugated to a primary antibody include, but are not limited to, Fluorescein, Rhodamine, Texas Red, VECTOR Red, ELF (Enzyme-Labeled Fluorescence), Cy2, Cy3, Cy3.5, Cy5, Cy7, Fluor X, Calcein, Calcein-AM, CRYPTOFLUOR, Orange (42 kDa), Tangerine (35 kDa), Gold (31 kDa), Red (42 kDa), Crimson (40 kDa), BHMP, BHDMAP, Br-Oregon, Lucifer Yellow, Alexa dye family, N-[6-(7-nitrobenz-2-oxa-1,3-diazol-4-yl)amino]caproyl] (NBD), BODIPY, boron dipyrromethene difluoride, Oregon Green, MITOTRACKER Red, Phycoerythrin, Phycobiliproteins BPE (240 kDa) RPE (240 kDa) CPC (264 kDa) APC (Allophycocyanin, 104 kDa), Spectrum Blue, Spectrum Aqua, Spectrum Green, Spectrum Gold, Spectrum Orange, Spectrum Red, Infra-Red (IR) Dyes, Cyclic GDP-Ribose (cGDPR), Calcofluor White, Lissamine, Umbelliferone, Tyrosine or Tryptophan.

In the method of the invention, the expression of at least one marker for T cell activity is detected in the PBMC by flow cytometry. The marker preferably is a marker for cytotoxic T cell activity. An additional marker for T cell activity can be a cytokine The term “cytokine” or “cytokines” as used herein refers to the general class of biological molecules which effect/affect cells of the immune system. The definition is meant to include, but is not limited to, those biological molecules that act locally or may circulate in the blood. Exemplary cytokines include but are not limited to interferons, interleukins, tumor necrosis factors and various colony stimulating factors. A T cell specific cytokine is herein understood to be a cytokine that is expressed in a T cell such as a CD4⁺ T cell and a CD8⁺ T cell. Alternatively, the marker for T cell activity, the expression of which is detected, is not a cytokine but a marker that otherwise indicates T cell activity, e.g. a marker for cytotoxic T cell activity such as a marker for T cell degranulation following antigenic stimulation.

In another embodiment of the method of the invention, the type of T cell expressing the marker for T cell activity is detected. Preferably, the method of the invention detects whether a given marker for T cell activity is expressed in a type of T cell that is at least one of a CD4⁺ cell and a CD8⁺ cell. More preferably, the method of the invention (simultaneously) detects whether a given marker for T cell activity is expressed in CD4⁺ T cell and whether the marker is expressed in a CD8⁺ T cell.

In one embodiment of the method of the invention, the marker for T cell activity—the expression of which is detected in the method—is a marker selected from the group consisting of CD107a, CD107b, IFN-γ, IL-2, IL-10 and TNF-α.

The CD107 molecule, consisting of CD107a and CD107b (LAMP-1/LAMP-2), is transiently expressed at the surface of T cells during T cell degranulation following antigenic stimulation (Betts et al., 2004, Meth. Cell Biol., 75:497-512). Expression of CD107 directly relates to the extrusion of cytotoxic compounds as part of the cytotoxic response of T cells. The CD107 molecule is present in the membrane of cytotoxic granules in the cell. At the moment of extrusion, the CD107 molecule is exposed at the outside of the cell. By adding labeled antibodies to the cell culture, the CD107 molecule will be labeled and visualized by flow cytometry. Consequently, expression of CD107 is the resultant of CD107 production and necessarily expression of the molecule at the cell membrane. In the method of the invention, expression of CD107 as a marker for T cell activity can be detected by detection of at least one of CD107a and CD107b.

CD107a (Cluster of Differentiation 107a) and CD107b (Cluster of Differentiation 107b) are also known as Lysosomal-associated membrane proteins 1 and 2 (LAMP1 and LAMP2), respectively (Chang et al., 2003, J. Biol. Regul. Homeost. Agents 16 (2): 147-151). Human CD107a is herein understood to be a glycoprotein having the amino acid sequence with NCBI accession no. NP_{—}005552 (version 3 Sep. 2009) or an allelic variant thereof. Human CD107b is herein understood to be a glycoprotein having the amino acid sequence with NCBI accession no. NP_{—}002285 (version 5 Oct. 2009) or an allelic variant thereof. CD107a and b have been implicated to present carbohydrate ligands to selectins and the proteins shuttle between lysosomes, endosomes, and the plasma membrane.

IFN-γ or interferon-gamma (IFN-γ) is a dimerized soluble cytokine that is the only member of the type II class of interferons (Gray et al., 1982, Nature 298 (5877): 859-863). IFN-γ is a cytokine which is critical for innate and adaptive immunity against viral and intracellular bacterial infections and for tumor control. IFN-γ is produced predominantly by natural killer (NK) and natural killer T (NKT) cells as part of the innate immune response, and by CD4⁺ and CD8⁺ cytotoxic T lymphocyte (CTL) effector T cells once antigen-specific immunity develops. Human IFN-γ is herein understood to be a protein having the amino acid sequence with NCBI accession no. NP_{—}000610 (version 28 Sep. 2009) or an allelic variant thereof.

IL-2, interleukin-2 is an interleukin that is instrumental in the body's natural response to microbial infection. Antigen binding to the T cell receptor (TCR) stimulates the secretion of IL-2 (Stern and Smith, 1986, Science. 233: 203-206). Human IL-2 is herein understood to be a protein having the amino acid sequence with NCBI accession no. NP_{—}000577 (version 28 Sep. 2009) or an allelic variant thereof.

IL-10, interleukin-10, plays a major role in suppressing immune and inflammatory responses by inhibiting the production of proinflammatory cytokines. It was reported that IL-10 produced by antiviral CD8⁺ and CD4⁺ effector T cells in the infected periphery during acute virus infection regulates the inflammatory response during acute influenza infection (Sun, 2009, Nature Medicine, 15(3):277-84). Human IL-10 is herein understood to be a protein having the amino acid sequence with NCBI accession no. CAG46825 (version 16 Octrober 2008) or an allelic variant thereof.

TNF-α, tumor necrosis factor-alpha (cachexin or cachectin) is a cytokine involved in systemic inflammation and is a member of a group of cytokines that stimulate the acute phase reaction. Human TNF-α is herein understood to be a protein having the amino acid sequence with NCBI accession no. NP_{—}000585 (version 28 Sep. 2009) or an allelic variant thereof.

Fluorescently labeled antibodies against markers, cytokines and differentiation antigens for use in the method of the present invention are commercially available from sources. Examples thereof are e.g. indicated in the Examples herein.

The method of the invention may be used for detecting the presence in a subject of an immune response against any virus including e.g. Orthomyxoviridae such as influenza virus; Retroviridae such as Human Immunodeficiency virus (HIV); a rubellavirus; paramyxoviridae such as parainfluenza viruses, measles, mumps, respiratory syncytial virus, human metapneumovirus; flaviviridae such as yellow fever virus, dengue virus, Hepatitis C Virus (HCV), Japanese Encephalitis Virus (JEV), tick-borne encephalitis, St. Louis encephalitis or West Nile virus; Herpesviridae such as Herpes Simplex virus, cytomegalovirus, Epstein-Barr virus; Bunyaviridae; Arenaviridae; Hantaviridae such as Hantaan; Coronaviridae; Papovaviridae such as human Papillomavirus; Rhabdoviridae such as rabies virus; Coronaviridae such as human coronavirus; Alphaviridae, Arteriviridae, filoviridae such as Ebolavirus, Arenaviridae, poxyiridae such as smallpox virus, and African swine fever virus.

In a preferred embodiment, the method of the invention is used for detecting the presence in a subject of an immune response against an influenza virus. Thus, in a preferred method according to the invention the inactivated virus is an inactivated influenza virus. The influenza virus may be an influenzavirus of the genera Influenzavirus A, B or C, of which A is most preferred. Subtypes of influenza A virus against which immune responses are preferably detected in the method of the invention include H1N1, H2N2, H3N2, H5N1, H7N7, H1N2, H9N2, H7N2, H7N3 and H10N7, including the 2009 pandemic swine origin influenza A (H1N1) virus (SOIV, or H1N1v).

The method of the invention may advantageously be used for phenotyping the immune response against the virus. In particular the T cell response may be phenotyped with respect to the number the type and the intensity of effector functions per cell as well as expression of distinct markers (by the cells).

In the method of the invention, the PBMCs are from a mammalian subject. The subject from which the PBMCs are obtained can be a non-human test animal, such as e.g. a mouse, rat, cotton rat (Sigmodon sp.) rabbit, non-human primate, or a ferret (e.g. Mustela putorius furo). Alternatively, the subject from which the PBMCs are obtained can be a human subject In one embodiment of the method of the invention, the PBMCs are from a human subject and the inactivated virus is an inactivated form of a virus that is able to infect humans and/or that is suspected thereof.

In the method of the invention, the amount of inactivated virus that is added to the PBMCs is at least sufficient to induce a (cellular) immune response as may be determined by the expression of a marker as described herein. The amount of inactivated virus to be added to the PBMCs is most conveniently related to the amount of virus prior to its activation. Thus, the amount of inactivated virus that is added to the PBMCs is the amount that corresponds to (is equal to) the amount of the virus prior to activation that would produce an M.O.I. of at least about 0.1, 0.2, 0.5, 1, or 2, preferably the M.O.I. is no more than about 10 or 5, more preferably about 1-3, most preferably an M.O.I. of about 2.

In a further embodiment of the method of the invention, the detection of expression of an intracellular marker may be improved by using monensin (e.g. Golgi-stop, BD Biosciences), an inhibitor of intracellular protein transport causing accumulation of proteins in the endoplasmic reticulum. Monensin is preferably added to the cultured PBMCs in the last hours prior to detection, e.g. during the last 20, 16, 12 or 8 hours of culture. For detection of expression of intracellular markers PBMCs will usually further be fixed and permeabilized by methods known in the art per se.

In a second aspect, the invention relates to a method for detecting a T cell response against a virus in a subject, wherein the method comprises the steps of: a) incubating PBMCs from the subject with inactivated virus; and, b) detection of expression of CD107 in the PBMCs. The method essentially is a method that is performed as described above except that in this aspect of the invention at least the expression of CD107 is determination in the PBMCs, whereas CD107 expression need not necessarily be determined by flow cytometry. Thus, in this second aspect of the invention CD107 expression in PBMCs stimulated with inactivated virus may be determined in a variety of methods known the skilled person. As indicated above expression of CD107 may be detected by detection of expression of at least one of CD107a and CD107b. It is further understood that in the various aspects of the invention CD107 expression may be detected in the cells as well as on the cells (surface).

As used herein, the term “expression” (e.g. of CD107 or another marker), when used in connection with detecting the expression of a gene, can refer to detecting transcription of the gene (i.e., detecting mRNA levels) and/or to detecting translation of the gene (detecting the protein produced). To detect expression of a gene refers to the act of actively determining whether a gene is expressed or not. This can include determining whether the gene expression is upregulated as compared to a control, downregulated as compared to a control, or unchanged as compared to a control. Therefore, the step of detecting expression does not require that expression of the gene actually is upregulated or downregulated, but rather, can also include detecting that the expression of the gene has not changed (i.e., detecting no expression of the gene or no change in expression of the gene). Expression of transcripts and/or proteins is measured by any of a variety of known methods in the art. For RNA expression, methods include but are not limited to: extraction of cellular mRNA and Northern blotting using labeled probes that hybridize to transcripts encoding all or part of the gene; amplification of mRNA using gene-specific primers, polymerase chain reaction (PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding the gene on any of a variety of surfaces; in situ hybridization; and detection of a reporter gene. Methods to measure protein expression levels generally include, but are not limited to: Western blot, immunoblot, enzyme-linked immunosorbant assay (ELISA), enzyme-linked immunosorbent spot (ELISPOT) or FluoroSpot assay, radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry.

In one embodiment of the method of the invention, in addition to detection of CD107 expression, optionally expression of further markers can be detected, such as e.g. one or more of IFN-γ, IL-2, IL-10 and TNF-α.

In a further aspect the invention pertains to a “kit” containing elements for use in the methods of the invention. Such a kit may comprise a carrier to receive therein one or more containers, such as tubes or vials. The kit may comprise inactivated virus as defined above, unlabeled or labeled antibodies for detection of markers, cytokines and/or differentiation antigens and others reagents mentioned herein such as e.g. culture media, monensin, reagents for fixing and permeabilization of cells and flow cytometry reagents. In a preferred embodiment, the kit at least comprises at least a preparation of inactivated virus and an antibody for detection of a marker, a cytokines and/or a differentiation antigen. The reagents may be present in lyophilized form, or in an appropriate buffer The kit may also contain any other component necessary for carrying out the present invention, such as means for obtaining PBMCs from subjects, buffers, pipettes, microtiter plates and written instructions. Such other components for the kits of the invention are known per se.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

The following examples are offered for illustrative purposes only, and are not intended to limit the scope of the present invention in any way.

### DESCRIPTION OF THE FIGURES

FIG. 1. Flow cytometry analysis of human PBMCs stimulated with mock culture supernatant as negative control, or with Staphylococcus enterotoxin B (SEB) as positive control, with live A/H3N2 virus (A/Wisconsin/67/2005; Live flu), or with A/H3N2 virus (A/Wisconsin/67/2005) inactivated with heat-treatment (H.I. flu), with formalin-treatment (F.I. flu), or with BPL-treatment (BPL. I. flu). IFN-γ (a), TNF-α (b), CD40L (c) and CD107 (d) expression was measured 24 hours, 72 hours and 6 days post infection/stimulation in CD8⁺ T cells as indicated. Virus inactivation was verified by three 10-day consecutive cultures of the inactivated preps on MDCK cells, without occurrence of any cyto-pathologic effect on the cells.

FIG. 2. Flow cytometry analysis of human PBMCs stimulated with live- or formalin inactivated (FI)-A/H3N2 virus (A/Wisconsin/67/2005) or with formalin-treated mock culture supernatant as negative control. IFN-γ (a and b), TNF-α (c and d), CD107a (e and f) and IL-2 (g and h) expression was measured at 24, 48, 72, and 168 hours post infection in CD4⁺ (a, c, e, and g) and CD8⁺ (b, d, f, and h) T cells as indicated. ▪ Mock; ▾ H3N2 virus; ♦ FI H3N2 virus.

FIG. 3. Flow cytometry analysis of human PBMCs stimulated with live- or FI-A/H3N2 virus, with formalin-treated mock culture supernatant as negative control, or with Staphylococcus enterotoxin B (SEB) as positive control. IFN-γ (a), TNF-α (b), CD107a (c) and IL-2 (d) expression was measured 72 hours post infection in CD4⁺ and CD8⁺ T cells as indicated.

FIG. 4. Flow cytometry analysis of human PBMCs from two recently SOIV A/H1N1-infected individuals (donors A and B) stimulated with formalin inactivated pandemic SOIV A/H1N1(A/Paris/2590/2009) virus (FI SOIV H1N1), seasonal live- or FI-A/H1N1 (A/New Calcdonia/20/99) virus live- or FI-A/H3N2 virus, formalin-treated mock culture supernatant as negative control, or with Staphylococcus enterotoxin B (SEB) as positive control. CD107a (a and b) and IL-2 (c and d) expression was measured 72 hours post infection in CD4⁺ (a and c) and CD8⁺ (b and d) T cells as indicated.

FIG. 5. Flow cytometry analysis of human PBMCs from donor MB-126 stimulated with formalin inactivated (FI)-A/H1N1 virus (A/New Calcdonia//20/99) and stained with an antibody specific for IL-10, or an isotype-matched antibody as a negative control. Expression was measured at 18, 36, 54 and 72 hours post stimulation in CD4⁺ (panel A) and CD8⁺ T (panel B) cells as indicated.

### EXAMPLES

### 1. Example 1

### 1.1. Materials & Methods

### 1.1.1 Inclusion of Donors and Isolation of PBMC

Buffy coats of healthy individuals were retrieved from Sanquin Blood Bank North West Region in accordance with the human experimental guidelines (project number S03.0015-X). In addition, peripheral blood mononuclear cells (PBMC) were retrieved from two previously healthy individuals (51 year old female, 55 year old male) with laboratory confirmed influenza A(H1N1)v infection, 13 and 19 days after start of symptoms, respectively. All participants provided written informed consent before the start of the study. The study was approved by the by the Medical Ethical Committee of the Utrecht University Medical Center. Human PBMC were isolated by density centrifugation and were cryopreserved at −135° C. in 90% FCS (Hyclone, Logan, Utah)/10% DMSO (Sigma-Aldrich, St Louis, USA) until analysis.

### 1.1.2 Influenza Virus

The seasonal influenza virus strains H3N2 A/Wisconsin/67/2005 and H1N1 A/New Calcdonia/20/99 as well as the SOIV H1N1 A/Paris/2590/2009 strain were generously provided by Dr. S. van der Werf (Institute Pasteur, Paris, France). All virus strains were produced by infecting MDCK cells. As negative control (mock) medium of uninfected MDCK cells was used.

### 1.1.3 Virus Inactivation

### 1.1.3.1 Formalin Inactivation

Influenza virus culture supernatants were incubated for 18 hours at 37° C. with 0.02% v/v formalin (Formalin 37%, Merck) in PBS. Immediately after inactivation, formalin was removed by dialysis using DispoDialyzers MWCO 10 kDa (Spectrum Laboratory Inc, Rancho Dominguez, USA), to prevent affecting the virologic and immune assays. The samples were stored at −80° C. until analysis.

### 1.1.3.2 Beta-Propiolactone (BPL) Inactivation

Influenza virus culture supernatants were incubated for 18 hours at 4° C. in a 1:1060 dilution of BPL (BPL, 5 ml flask 98% solution, Acros, www.acros.com) in 0.1 M phosphate buffer at pH 7.3. Next the BPL was hydrolysed by incubation at 37° C. for 2 hours. The samples were stored at −80° C. until analysis.

### 1.1.3.2 Heat Inactivation

Influenza virus culture supernatants were incubated for 30 minutes at 65° C. The samples were stored at −80° C. until analysis.

### 1.1.4 PBMC Stimulation and Flow Cytometric Analysis

PBMC were infected at a multiplicity of infection (MOI) of 2 with live influenza virus or stimulated with an equal amount of inactivated virus and cultured in RPMI medium containing 10% FCS, P/S at 1*10⁶ cells per well of a 48-wells plate (Greiner Bio-one). Culture supernatant of mock-infected cells was inactivated with formalin and used as a negative control (mock). As a positive control, Staphylococcus enterotoxin B (SEB) was used. The cells were incubated for various times at 37° C. and 5% percent CO₂. Antibodies specific for CD107a PE (BD Biosciences, San Jose, USA) were added 16 hours before the end of culture. For detection of intracellular cytokine production, monensin (Golgi-stop, BD Biosciences) was added during the last 16 hours of culture. After incubation, PBMC were harvested, transferred to a V-bottom plate (Greiner BioOne) and washed with FACS buffer (PBS, 0.5% BSA, 5 mM EDTA). The cells were stained with CD4 Pacific blue (Biolegend, San Diego, USA), CD8 PerCP Cy5.5 (BD Biosciences) and LIVE/DEAD Fixable Dead Cell Stain (Invitrogen, Paisley, UK) for 30 min. at 4° C. in the dark. PBMC were washed once with 100 ul FACS buffer. Subsequently, the PBMC were fixed and permeabilized in CytoFix/CytoPerm solution (BD Biosciences) for 20 min. at 4° C. in the dark. PBMC were washed three times with 100 ul Perm/Wash buffer (BD Biosciences) and were stained with IFN-γ APC (BD Biosciences), IL-2 FITC (eBioscience, San Diego, USA), CD40L APC Alexa Fluor 750 (eBioscience) and TNF-α PeCy7 (BD Biosciences) for 30 min. at 4° C. in the dark. After staining, PBMC were washed two times with 100 ul Perm/Wash buffer and one time with 100 ul FACS buffer. PBMC were resuspended in 150 ul of FACS buffer and acquired directly using a FACS Canto II (BD Biosciences). At least 100.000 viable lymphocytes were acquired based on forward—side scatter characteristics and analysis of live-dead staining. The results were analyzed using FACSDiva software (BD Biosciences).

### 1.1.5 Validation of Inactivation of Virus

### 1.1.5.1 Inactivation Validation by Culturing on MDCK Cells

Both control and treated influenza virus culture supernatants were subjected to a maximum of three blind passages on MDCK cells. Therefore, confluent MDCK cells were incubated at 37° C. for 60 min with 50 μl culture supernatant and 5 ml infection medium in T25 cm²-flasks. After one hour the monolayer was rinsed with phosphate-buffered saline (PBS), overlaid with infection medium (2.5 μg/ml trypsin) and incubated at 37° C. for 10 days. After a ten-day incubation period, the flasks were subjected to one freeze-thaw cycle. In the absence of cpe, the harvested culture material was used as inoculum (50 μl) for a following passage on MDCK cells. All harvested culture supernatants were stored at −80° C.

### 1.1.5.2 Inactivation Validation by Haemagglutination Assay

Serial two-fold dilutions of live and inactivated influenza virus culture supernatants were prepared in PBS and incubated with 0.25% turkey erythrocytes in PBS at 4° C. After 60 minutes the haemagglutination titer, expressed as the reciprocal of the highest dilution producing complete haemagglutination, was read.

### 1.1.5.3 Inactivation Validation by Matrix PCR

Total nucleic acid (50 μl) was extracted from 200 μl influenza virus culture supernatant with the MagNA Pure LC total nucleic acid isolation kit, on a MagNA Pure LC (v3.0) extraction robot (Roche, Mannheim, Germany). For qualitative analysis of influenza negative sense genomic RNA in the culture materials, reverse transcriptase polymerase chain reaction (RT-PCR) was performed using Avian Myeloblastosis Virus Reverse Transcriptase (AMV-RT) (Promega, Wis., USA) and the sense matrix gene primer 5′ AAG ACC AAT CCT GTC ACC TCT GA 3′ (M-Fw) to generate copy DNA (cDNA). To detect positive sense viral RNA transcripts in the culture material, total RNA was transcribed in cDNA using rTth DNA polymerase (Applied Biosystems, CA, USA) and matrix gene antisense primer 5′ CAA AGC GTC TAC GCT GCA GTC C 3′ (M-Rv) followed by RNAse H (Roche) and RNase A (Sigma-Aldrich) treatment to remove all RNA. Subsequently, a 104 base-pair matrix gene fragment was amplified using the LightCycler® 480 realtime PCR system (Roche), LightCycler® TaqMan Master (Roche), primer pair M-Fw and M-Rv, and amplicon specific probe 5′ TTT GTG TTC ACG CTC ACC GTG CC 3′ labeled with FAM/BHQ-1®.

### 1.2 Results

1.2.1 A Comparison of Three Different Methods of Inactivation of Influenza and their Effect on T Cell Responses

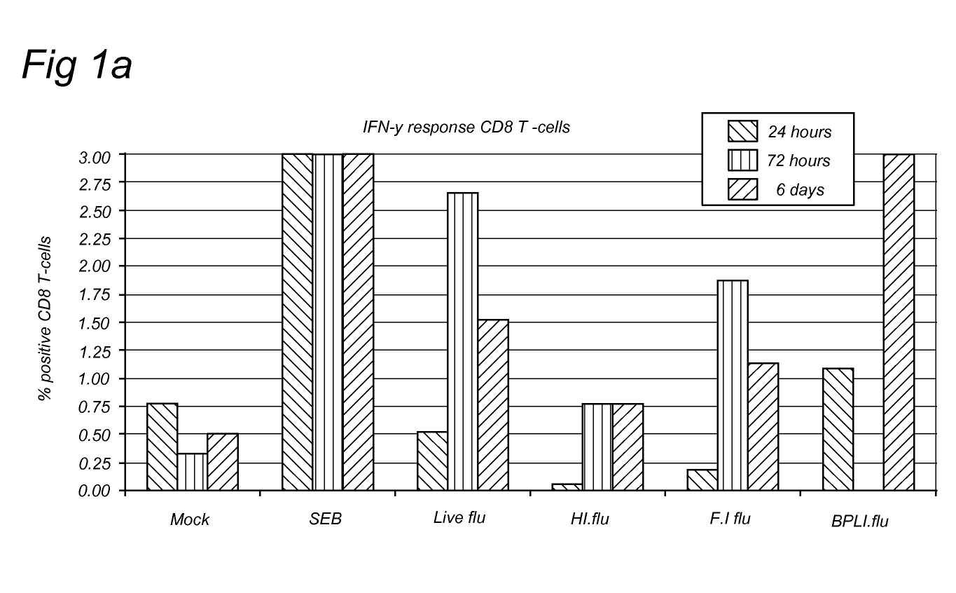

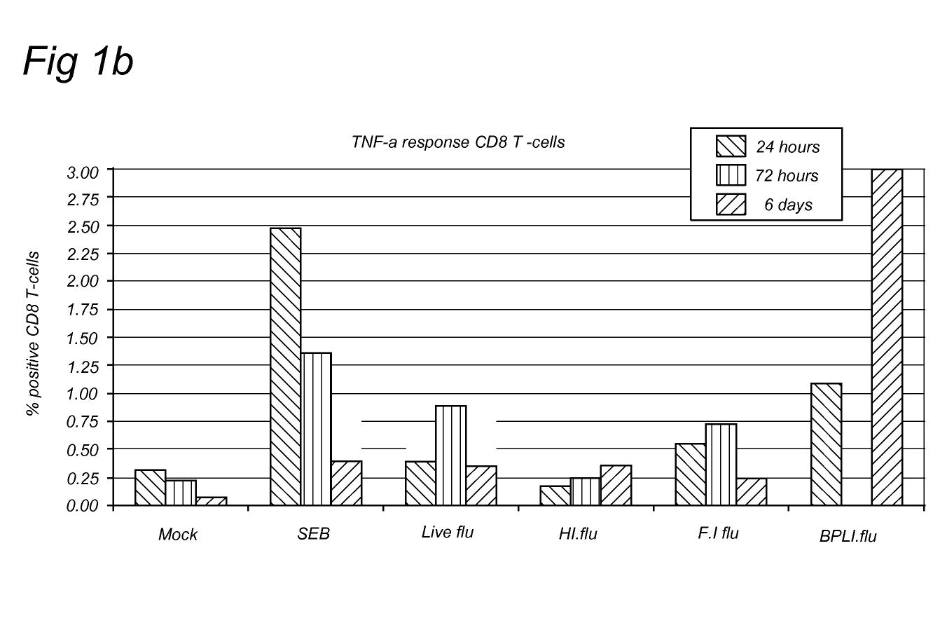

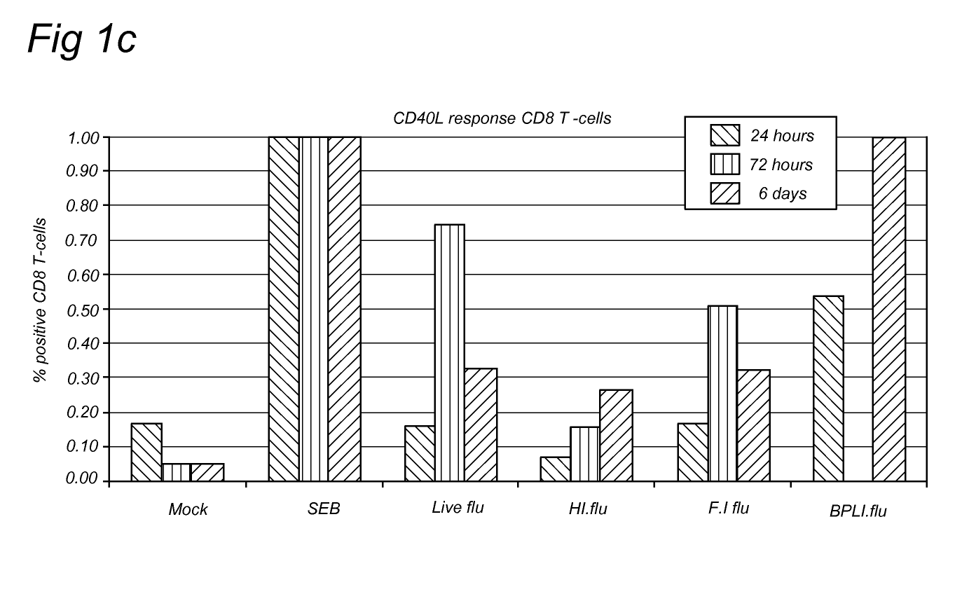

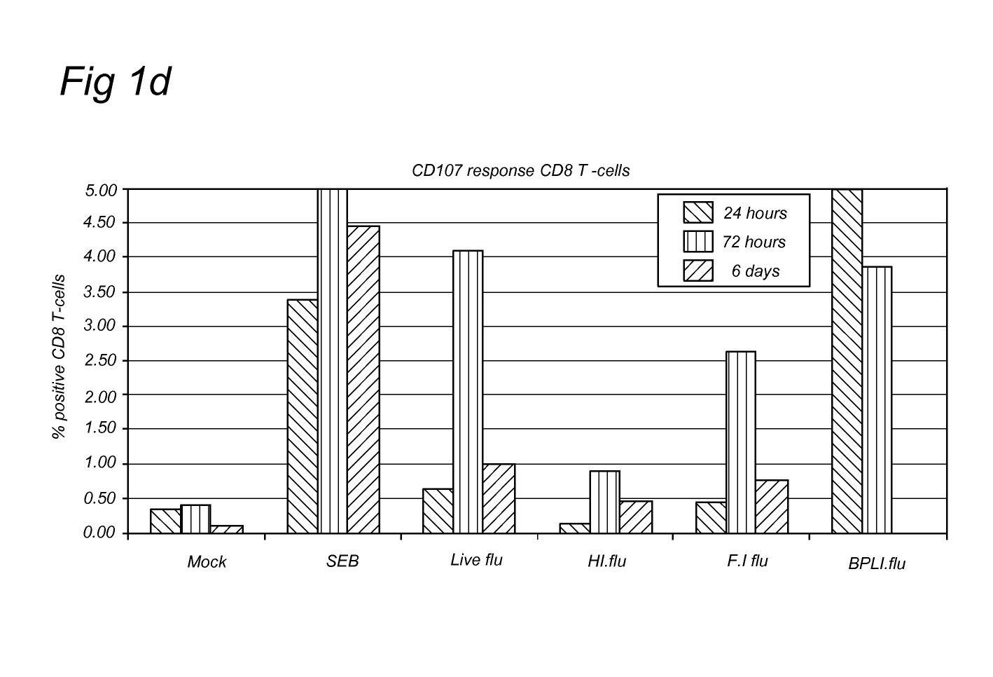

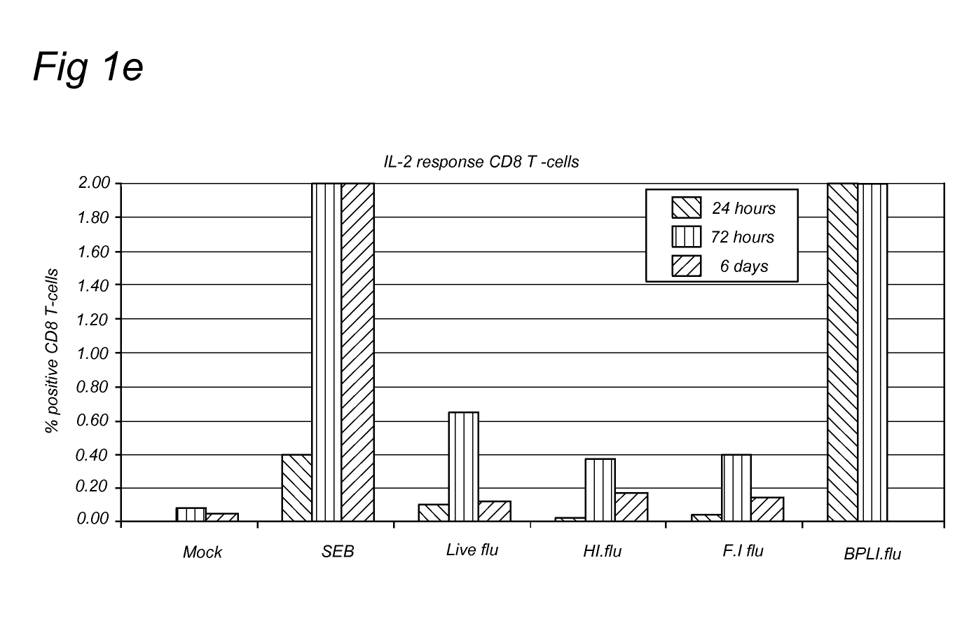

We first exploited various techniques for inactivation of influenza virus. Virus inactivated by various techniques was then used to stimulate PBMC and next CD8⁺ T cell responses were evaluated. The methods for viral inactivation included: heat inactivation, formalin inactivation and beta propiolactone (BPL) inactivation (see 1.1.3 above). CD8⁺ T cell responses obtained after stimulation with inactivated virus were compared with the response obtained with live virus stimulation. As a positive control stimulation with Staphylococcal exotoxin B (SEB) was used. IFN-γ, TNF-α, CD40L and CD107a and IL-2 responses were determined in CD4⁺ and CD8⁺ T cells. FIG. 1 shows that in CD8⁺ T cells BPL-inactivated virus gave very inconsistent responses, heat inactivated virus gave little or no responses, but formalin inactivated virus produced consistent CD8⁺ T cell responses of about 75% of the intensity obtained with live virus infection. Similar results were obtained for CD4⁺ T cells (data not shown).

### 1.2.2 T Cell Responses to Formalin-Inactivated Seasonal Influenza

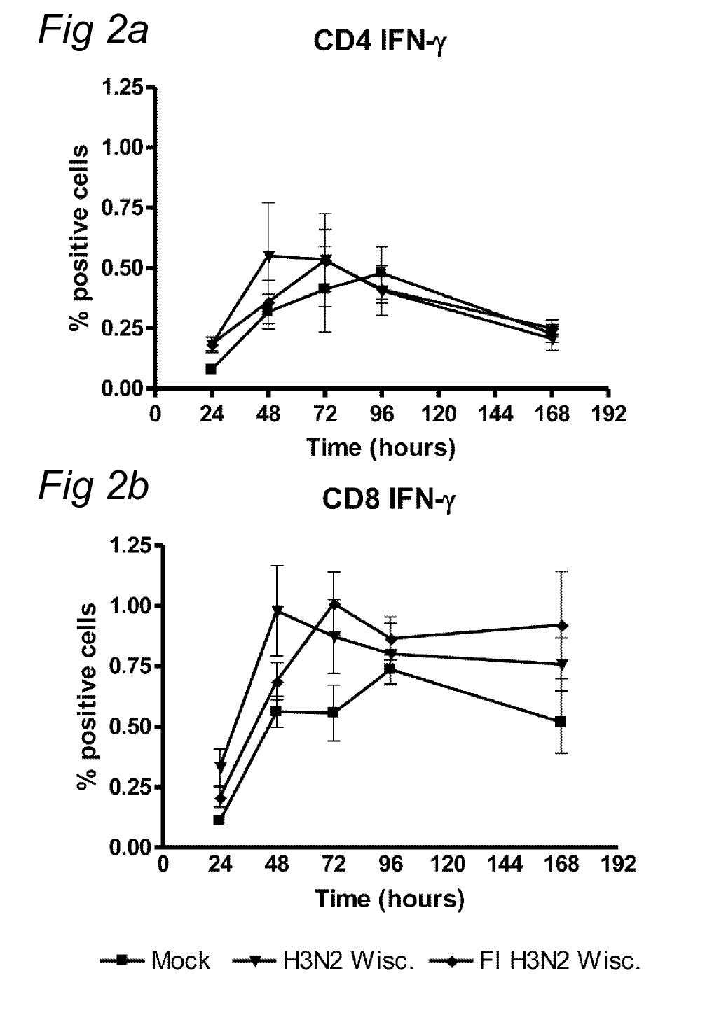

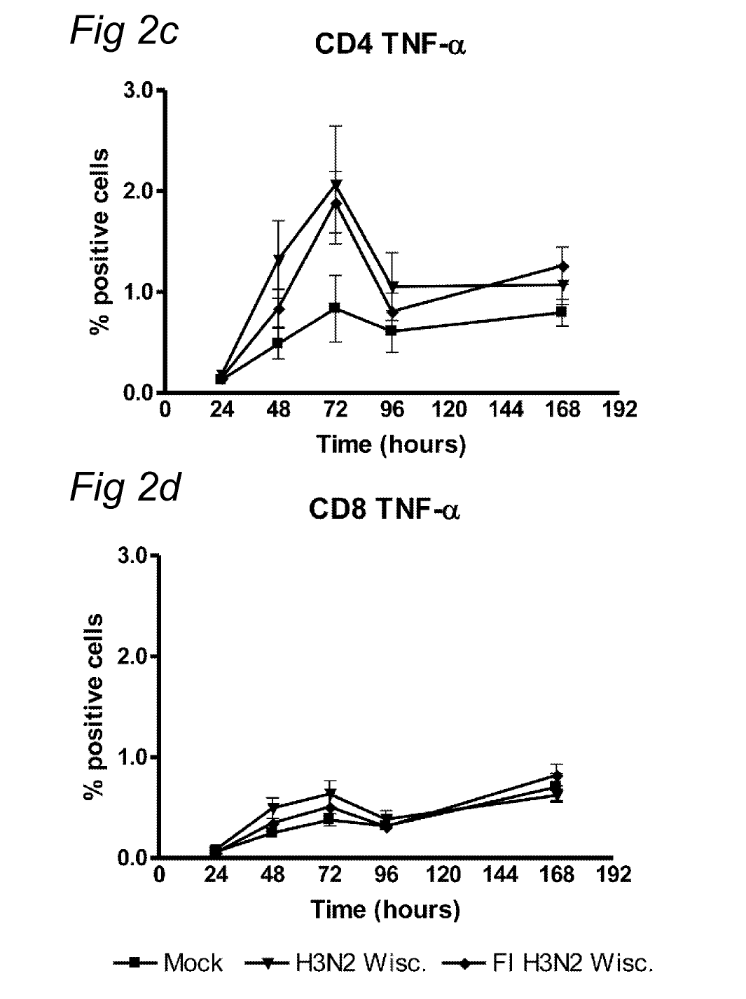

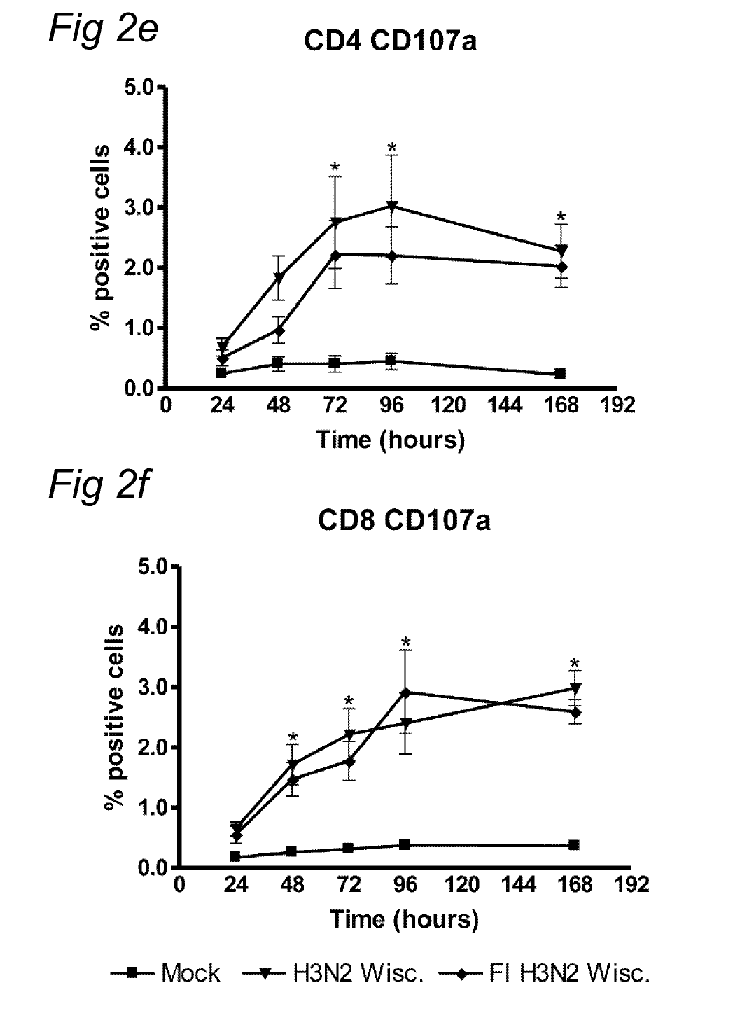

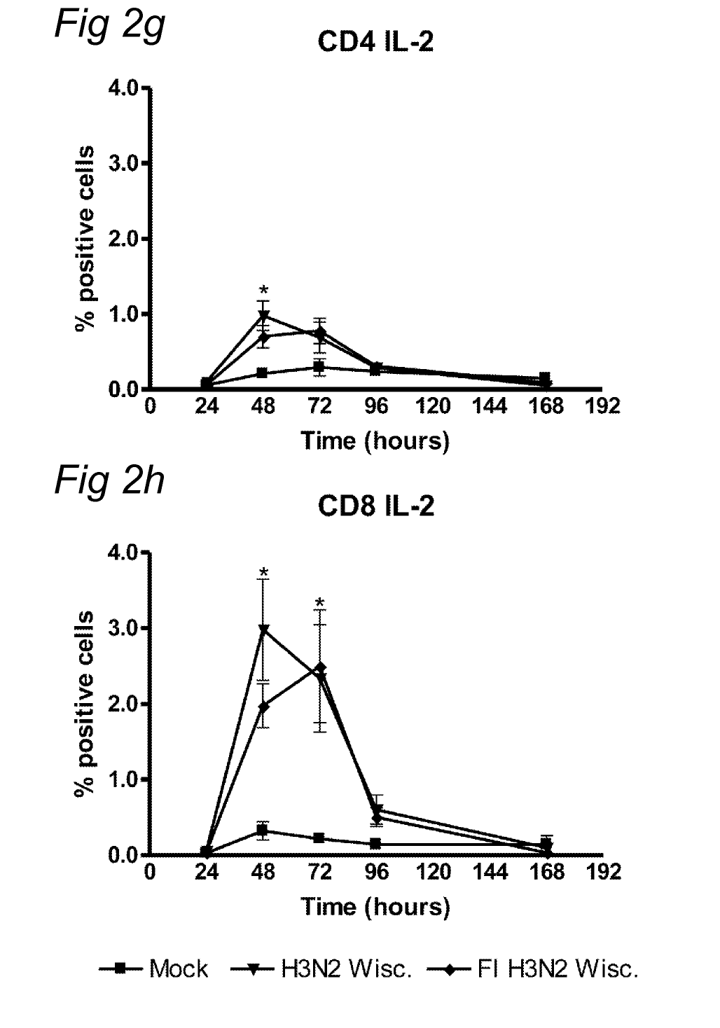

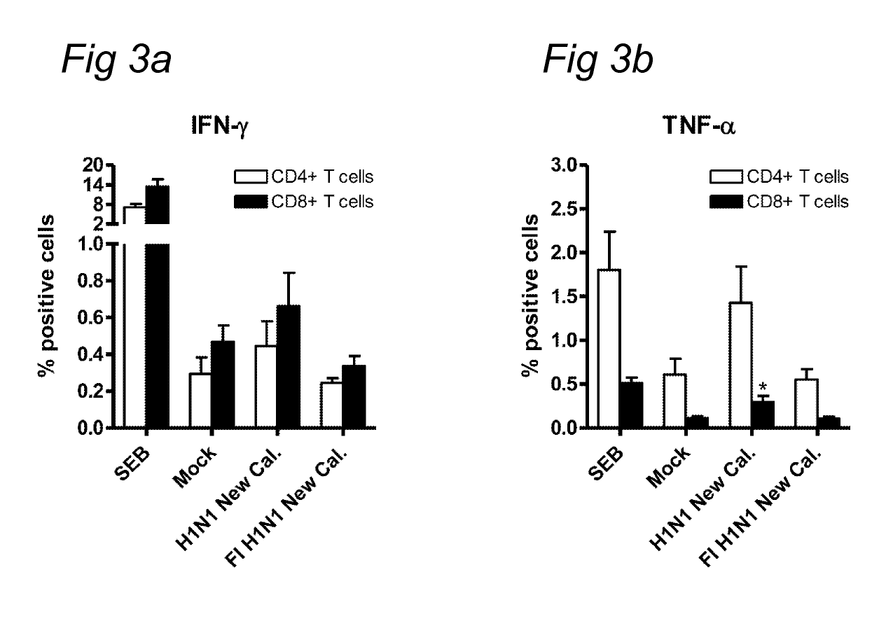

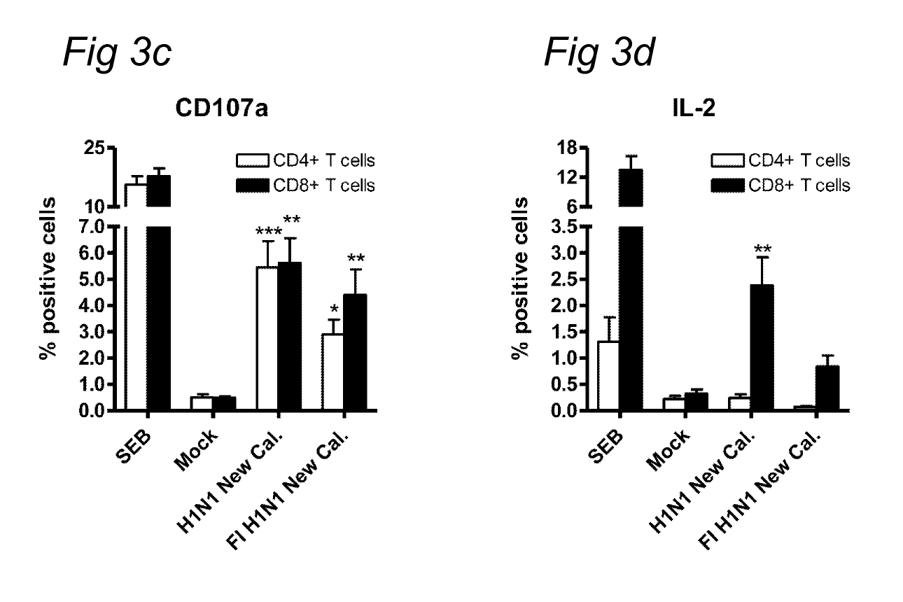

Treatment of influenza virus with formalin only marginally reduced haemagglutination and neuraminidase activity and showed the highest degree of HA and NA activity preservation. Therefore, formalin-inactivated (FI) influenza virus was used to determine whether T cell epitope antigenicity is preserved after formalin treatment. First, the optimal time point for detection of T cell responses was determined by incubating PBMCs with live- or FI-A/H3N2 virus (A/Wisconsin/67/2005) after which IFN-γ, TNF-α, CD107a and IL-2 expression was measured at 24, 48, 72, and 168 hours post infection (p.i.). Stimulation of PBMCs with formalin-treated mock was used to determine background expression of all markers. Stimulation of PBMCs with live A/H3N2 virus resulted in a significant increase in CD4⁺ and CD8⁺ T cells expressing CD107a and IL-2 and was highest between 48 hours-96 hours p.i. (FIG. 2). A similar expression pattern was observed in PBMCs stimulated with FI-A/H3N2 virus. However, the highest percentage of CD107a⁺ and IL-2⁺ cells was reached approximately 24 hours later with FI-A/H3N2 stimulation compared to live A/H3N2 stimulation. In addition, the percentages of CD107a⁺ and IL-2⁺ T cells induced by FI-A/H3N2 were lower as compared to the percentages induced by live H3N2 virus, but were still significantly higher than the percentages induced by mock stimulation. Similar to the induction of CD107a⁺ and IL-2⁺ T cells, stimulation of PBMCs with live- and FI-A/H3N2 induced a non-significant trend to increased IFN-γ⁺ and TNF-α⁺ T cells. In general, the detection of T cell responses to live- and FI-A/H3N2 stimulation was most optimal at 72 hrs p.i. and was used for further examination of other influenza subtypes. This time point was used for further examination of the T cell response to a live- and FI-A/H1N1 subtype influenza virus (A/New Calcdonia/20/99) (FIG. 3). The A/H1N1 virus also induced a significant number of CD107a⁺ and IL-2⁺ T cells, both after live- and FI-A/H1N1 stimulation. Here again, percentages of CD107a⁺ and IL-2⁺ T cells induced by FI-A/H1N1 were significantly higher than the percentages induced by mock infection. Comparable to the A/H3N2 virus, the induction of IFN-γ⁺ and TNF-α+ T cells by FI-A/H1N1 stimulation was not significantly induced.

Together, these data demonstrate that besides HA and NA activity, the T cell epitope antigenicity of seasonal influenza viruses is highly preserved after formalin treatment.

### 2.1.3 T Cell Responses to Formalin-Inactivated Pandemic SOIV A/H1N1

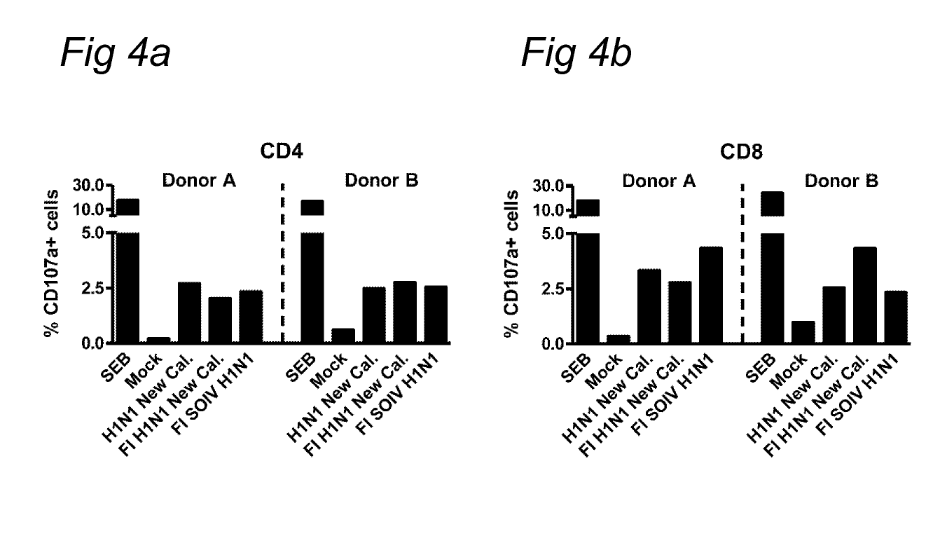

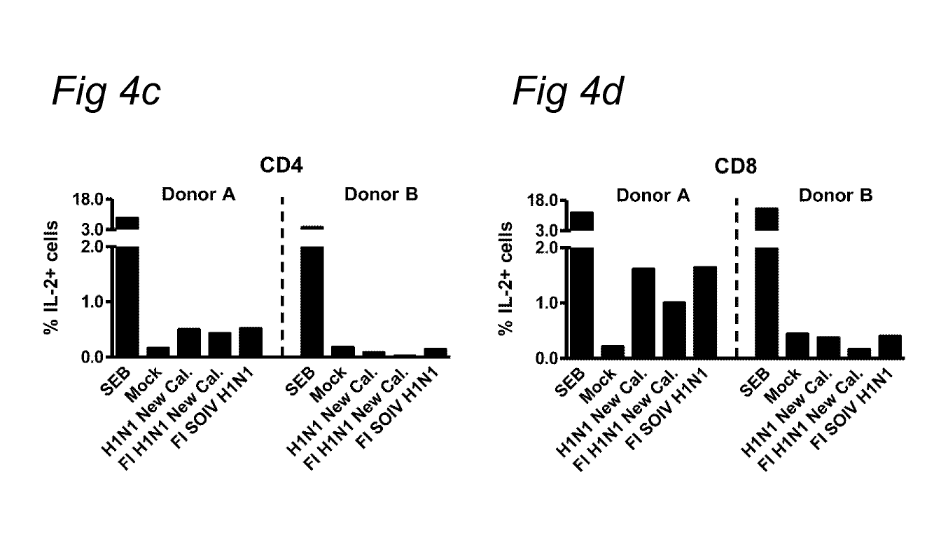

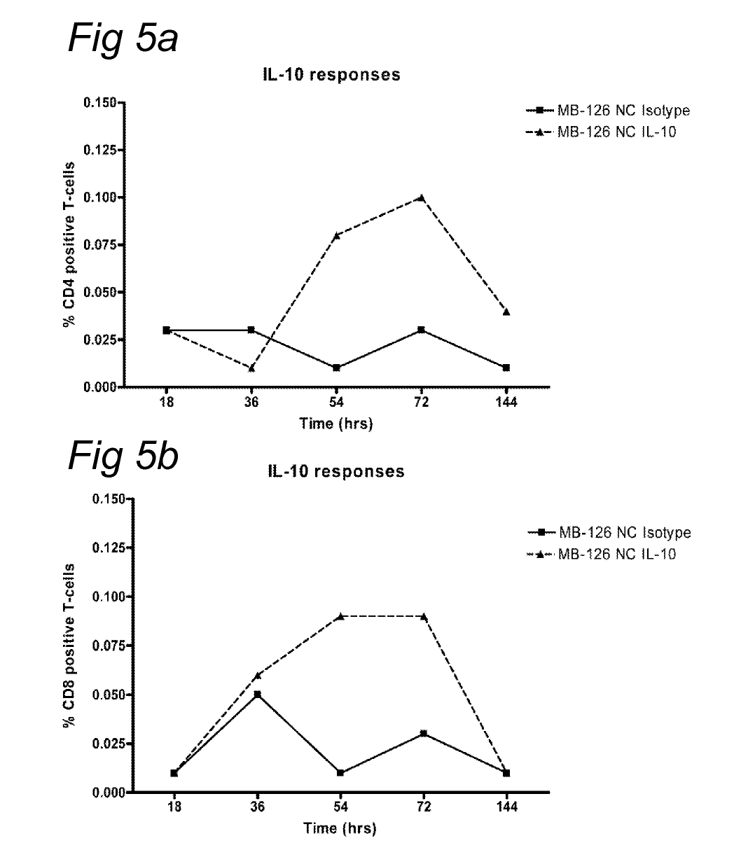

Since formalin treatment effectively inactivated seasonal influenza virus, but preserved T cell epitope antigenicity, we wished to determine whether T cell epitope antigenicity of the pandemic SOIV A/H1N1 virus was also preserved after inactivation by formalin treatment. To determine T cell responses to FI-SOIV A/H1N1 (A/Paris/2590/2009) we used isolated PBMCs from two recently SOIV A/H1N1-infected individuals to ensure the presence of significant numbers of SOIV A/H1N1-specific T cells and determined the induction of CD107a⁺ and IL-2⁺ T cells. As a control, PBMCs were stimulated with FI-mock, seasonal live- or FI-A/H1N1 (A/New Calcdonia/20/99) virus. In vitro stimulation of PBMCs from the recently SOIV A/H1N1-infected individuals with FI-SOIV A/H1N1 demonstrated in both donors a significant increase in the percentage of CD107a+ T cells in the CD4⁺ and CD8⁺ T cell subsets (FIG. 4). The seasonal live- and FI-A/H1N1 viruses induced a similar increase in CD107a+ cells. Furthermore, live A/H1N1, FI-A/H1N1 and FI-SOIV A/H1N1 stimulation induced IL-2⁺ T cells in PBMCs from donor A and was most apparent in the CD8⁺ T cell subset. However, stimulation of PBMCs from donor B with live A/H1N1, FI-A/H1N1 or FI-SOIV A/H1N1 did not induce IL2⁺ T cells in both the CD4⁺ and CD8⁺ T cell subsets, indicating some donor variability in the T cell response.

Altogether, these data demonstrate that the pandemic SOIV A/H1N1 virus retains T cell stimulatory capacity after formalin treatment, indicating that T cell epitope antigenicity is also preserved for this pandemic influenza virus.

### REFERENCE LIST

1. Goldstein, M. A. & Tauraso, N. M. (1970) “Effect of formalin, beta-propiolactone, merthiolate, and ultraviolet light upon influenza virus infectivity chicken cell agglutination, hemagglutination, and antigenicity” Appl. Microbiol. 19: 290-294.
2. Geeraedts, F., Goutagny, N., Hornung, V., Severa, M., de, H. A., Pool, J., Wilschut, J., Fitzgerald, K. A., & Huckriede, A. (2008) “Superior immunogenicity of inactivated whole virus H5N1 influenza vaccine is primarily controlled by Toll-like receptor signalling” PLoS. Pathog. 4: e1000138.
3. Bachmann, M. F., Bast, C., Hengartner, H., & Zinkernagel, R. M. (1994) “Immunogenicity of a viral model vaccine after different inactivation procedures” Med. Microbiol. Immunol. 183: 95-104.
4. Jonas E. Salk (1944) “A Simplified Procedure for Titrating Hemagglutinating Capacity of Influenza-Virus and the Corresponding Antibody” The Journal of Immunology 49: 87-98.
5. Van Deusen, R. A., Hinshaw, V. S., Senne, D. A., & Pellacani, D. (1983) “Micro neuraminidase-inhibition assay for classification of influenza A virus neuraminidases” Avian Dis. 27: 745-750.
6. Mbawuike, I., Zang, Y., & Couch, R. B. (2007) “Humoral and cell-mediated immune responses of humans to inactivated influenza vaccine with or without QS21 adjuvant” Vaccine 25: 3263-3269.
7. Lalvani, A., Brookes, R., Hambleton, S., Britton, W. J., Hill, A. V., & McMichael, A. J. (1997) “Rapid effector function in CD8+ memory T cells” J. Exp. Med. 186: 859-865.
8. Seder, R. A., Darrah, P. A., & Roederer, M. (2008) “T-cell quality in memory and protection: implications for vaccine design” Nature Reviews Immunology 8: 247-258|doi:10.1038/nri2274
9. Rutebemberwa et al. (2007) “Evaluation of aldrithiol-2-inactivated preparations of HIV type 1 subtypes A, B, and D as reagents to monitor T cell responses” AIDS Res. Hum. Retroviruses 23: 532-542.
10. Skarsvik et al. (2006) “Decreased in vitro type 1 immune response against Coxsackie virus B4 in children with type 1 diabetes.” Diabetes 55: 996-1003.
11. Betts et al. (2006) “HIV nonprogressors preferentially maintain highly functional HIV-specific CD8(+) T cells” Blood 107: 4781-4789.
12. Lambre, C. R., H. Terzidis, A. Greffard, and R. G. Webster. 1990. Measurement of anti-influenza neuraminidase antibody using a peroxidase-linked lectin and microtitre plates coated with natural substrates. J. Immunol. Methods 135:49-57.

1. A method for detecting a T cell response against a virus in a subject, comprising:
(a) incubating PBMCs from the subject with formalin-inactivated virus;
(b) detecting expression of at least one marker for T cell activity by flow cytometry.

2. The method according to claim 1, wherein the expression of at least two different markers for T cell activity is detected.

3. The method according to claim 1, further comprising detecting the type of T cell expressing at least one marker for T cell activity.

4. The method according to claim 3, wherein the type of T cell is at least one of a CD4⁺ cell and a CD8⁺ cell.

5. The method according to claim 1, wherein the at least one marker is selected from the group consisting of CD107a, CD107b, IFN-γ, IL-2, IL-10 and TNF-α.

6. The method according to claim 1, wherein the inactivated virus is an inactivated influenza virus.

7. The method according to claim 6, wherein the inactivated influenza virus is obtained by treatment of the virus that produces the same degree of viral inactivation as a treatment for at least 18 hours at 37° C. with at least 0.02% v/v formalin.

8. The method according to claim 1, wherein the incubating is between about 24 and 96 hours.

9. The method according to claim 8, wherein the incubating is between about 60 and 84 hours.

10. The method according to claim 1, wherein the amount of inactivated virus corresponds to an amount of virus prior to activation that produces an M.O.I. of about 1-3.

11. The method according to claim 1, wherein the PBMCs are from a human subject and wherein the inactivated virus is an inactivated form of a virus that is able or suspected to infect humans.

12. A method for detecting a T cell response against an virus in a subject, comprising:
(a) incubating PBMCs from the subject with inactivated virus;
(b) detecting expression of at least one of CD107a and CD107b in the PBMCs and,
(c) optionally detecting the expression of one or more further T cell specific cytokines.

13. The method according to claim 12, wherein the inactivated virus is an inactivated influenza virus.

14. A kit for detecting a T cell response against an virus, comprising a container with a formalin-inactivated preparation of the virus and a container with an antibody for detection of a marker for T cell activity.


SEQUENCE LISTING (3 sequences)

SEQ ID NO: 1 - DNA, 23 bp, Artificial Sequence
  Description of Artificial Sequence Synthetic primer
aagaccaatcctgtcacctctga

SEQ ID NO: 2 - DNA, 22 bp, Artificial Sequence
  Description of Artificial Sequence Synthetic primer
caaagcgtctacgctgcagtcc

SEQ ID NO: 3 - DNA, 23 bp, Artificial Sequence
  Description of Artificial Sequence Synthetic probe
tttgtgttcacgctcaccgtgcc